(19) United States
(12) Patent Application Publication
(10) Pub. No.: US 2025/0177651 A1
(43) Pub. Date: Jun. 5, 2025

(54) INSERTING ASSEMBLY AND RELATED AUTOMATED INJECTION SYSTEM

(71) Applicant: ALTEK BIOTECHNOLOGY CORPORATION, Hsinchu City (TW)

(72) Inventor: Yu-Cheng Huang, Hsinchu City (TW)

(21) Appl. No.: 18/527,373

(22) Filed: Dec. 4, 2023

(51) Int. Cl. A61M 5/20; A61M 5/32

(52) U.S. Cl. CPC A61M 5/2033; A61M 5/3287

(57) ABSTRACT

An inserting assembly is provided and includes a driving component, an actuating component, an abutting kit, a first ratchet component, a driven component and an inserting component. The actuating component is for biasing the driving component to rotate. The abutting kit is for engaging with the driving component at a first position for stopping the driving component from rotating from the first position to a second position. When the first ratchet component rotates, the first ratchet component drives the abutting kit to disengage from the driving component at the first position, such that the actuating component drives the driving component to rotate from the first position to the second position, so as to enable the driving component to drive the driven component and the inserting component to move together for achieving insertion of the inserting component. Besides, a related automated injection system is also provided.

### BACKGROUND OF THE INVENTION

### 1. Field of the Invention

The present invention relates to an inserting assembly and a related automated injection system, and more specifically, to an inserting assembly with simple structure and easy operation and a related automated injection system.

### 2. Description of the Prior Art

An automated injection system, e.g., an on-body injector, is a medical device designed for drug delivery. However, the conventional auto-injectors available in the markets are unable to meet requirements of simple structure and easy operation. Therefore, an improvement of the automated injection system is urgently needed.

### SUMMARY OF THE INVENTION

It is an objective of the present invention to provide an inserting assembly with simple structure and easy operation and a related automated injection system for solving the aforementioned problem.

In order to achieve the aforementioned objective, the present invention discloses an inserting assembly. The inserting assembly includes a driving component, an actuating component, an abutting kit, a first ratchet component, a driven component and an inserting component. The actuating component abuts against the driving component and is configured to bias the driving component to rotate around a rotating axis. The abutting kit is configured to engage with the driving component at a first position for stopping the driving component from rotating from the first position to a second position around the rotating axis. The first ratchet component is coaxially rotatable relative to the driving component. The first ratchet component drives the abutting kit to disengage from the driving component at the first position when the first ratchet component rotates along a first rotating direction, such that the actuating component drives the driving component to rotate from the first position to the second position. The driven component is movably engaged with the driving component. The driving component drives the driven component to move along an inserting direction when the actuating component drives the driving component from the first position to the second position. The inserting component is disposed on the driven component and movable along the inserting direction together with the driven component.

According to an embodiment of the present invention, the abutting kit is further configured to engage with the driving component at the second position for stopping the driving component from rotating away from the second position when the actuating component drives the driving component to rotate from the first position to the second position around the rotating axis.

According to an embodiment of the present invention, the inserting assembly further includes a second ratchet component coaxially rotatable relative to the driving component. The second ratchet component is resiliently deformed by the first ratchet component and not driven to rotate along the first rotating direction by the first ratchet component when the first ratchet component rotates along the first rotating direction, and the second ratchet component is driven by the first ratchet component to rotate along a second rotating direction opposite to the first rotating direction when the first ratchet component rotates along the second rotating direction.

According to an embodiment of the present invention, the second ratchet component drives the abutting kit to disengage from the driving component at the second position when the second ratchet component is driven by the first ratchet component to rotate along the second rotating direction, and the actuating component drives the driving component to rotate away from the second position around the rotating axis when the second ratchet component drives the abutting kit to disengage from the driving component at the second position.

According to an embodiment of the present invention, the driving component drives the driven component to move along a retracting direction when the actuating component drives the driving component to rotate away from the second position around the rotating axis, and the driven component drives the inserting component to move together with the driven component along the retracting direction when the driving component drives the driven component to move along the retracting direction.

According to an embodiment of the present invention, the driving component includes an inserting inclined surface and a retracting inclined surface. The inserting inclined surface and the retracting inclined surface are connected to each other. The inserting inclined surface is configured to abut against the driven component for driving the driven component to move along the inserting direction when the actuating component drives the driving component to rotate away from the first position and toward the second position around the rotating axis, and the retracting inclined surface is configured to abut against the driven component for driving the driven component to move along the retracting direction when the actuating component drives the driving component to rotate away from the second position and toward a third position around the rotating axis.

According to an embodiment of the present invention, the first ratchet component, the second ratchet component and the driving component are arranged along the rotating axis. The second ratchet component is located between the first ratchet component and the driving component, and the abutting kit is located at a position corresponding to an outer periphery of the first ratchet component, an outer periphery of the second ratchet component, and an outer periphery of the driving component.

According to an embodiment of the present invention, the abutting kit includes a first abutting portion and a second abutting portion. An end of the first abutting portion protrudes beyond an end of the second abutting portion along a direction from the second ratchet component to the first ratchet component. The first abutting portion is configured to cooperate with the driving component and the first ratchet component, and the second abutting portion is configured to cooperate with the driving component and the second ratchet component.

According to an embodiment of the present invention, the abutting kit further includes a connecting portion integrally connected between the first abutting portion and the second abutting portion, and the first abutting portion, the second abutting portion and the connecting portion are made of resilient material.

According to an embodiment of the present invention, the first abutting portion and the second abutting portion are separated from each other and made of resilient material.

According to an embodiment of the present invention, the abutting kit further includes a first recovering portion and a second recovering portion. The first recovering portion and the second recovering portion are made of resilient material and abut against the first abutting portion and the second abutting portion, respectively.

According to an embodiment of the present invention, the actuating component biases the driving component to rotate along a second rotating direction opposite to the first rotating direction around the rotating axis.

In order to achieve the aforementioned objective, the present invention further includes an inserting assembly. The inserting assembly includes a driving component, an actuating component, an abutting kit, a first ratchet component, a second ratchet component, a driven component and an inserting component. The actuating component abuts against the driving component and is configured to bias the driving component to rotate around a rotating axis. The abutting kit is configured to engage with the driving component. The first ratchet component is coaxially rotatable relative to the driving component. The first ratchet component drives the abutting kit to disengage from the driving component at a first position when the first ratchet component rotates along a first rotating direction, such that the actuating component drives the driving component to rotate from the first position to a second position around the rotating axis along a second rotating direction opposite to the first rotating direction, and the abutting kit engages with the driving component at the second position for stopping the driving component from rotating away from the second position along the second rotating direction when the actuating component drives the driving component to rotate from the first position to the second position along the second rotating direction. The second ratchet component is coaxially rotatable relative to the driving component. The second ratchet component is driven by the first ratchet component to rotate along the second rotating direction when the first ratchet component rotates along the second rotating direction. The second ratchet component drives the abutting kit to disengage from the driving component at the second position when the second ratchet component is driven by the first ratchet component to rotate along the second rotating direction, and the actuating component drives the driving component to rotate from the second position to a third position around the rotating axis along the second rotating direction when the second ratchet component drives the abutting kit to disengage from the driving component at the second position. The driven component is movably engaged with the driving component. The driving component drives the driven component to move along an inserting direction when the actuating component drives the driving component from the first position to the second position. The driving component drives the driven component to move along a retracting direction when the actuating component drives the driving component to rotate from the second position to the third position. The inserting component is disposed on the driven component and movable along the inserting direction or the retracting direction together with the driven component.

In order to achieve the aforementioned objective, the present invention further discloses an automated injection system. The automated injection system includes a case and an inserting assembly mounted on the case, and the inserting assembly includes a driving component, an actuating component, an abutting kit, a first ratchet component, a driven component and an inserting component. The actuating component abuts against the driving component and is configured to bias the driving component to rotate around a rotating axis. The abutting kit is configured to engage with the driving component at a first position for stopping the driving component from rotating from the first position to a second position around the rotating axis. The first ratchet component is coaxially rotatable relative to the driving component. The first ratchet component drives the abutting kit to disengage from the driving component at the first position when the first ratchet component rotates along a first rotating direction, such that the actuating component drives the driving component to rotate from the first position to the second position. The driven component is movably engaged with the driving component. The driving component drives the driven component to move along an inserting direction when the actuating component drives the driving component to rotate from the first position to the second position. The inserting component is disposed on the driven component and movable along the inserting direction together with the driven component.

In summary, the present invention can achieve the inserting component to move along the inserting direction and then along the retracting direction by rotating the first ratchet component along the first rotating direction and then along the second rotating direction only. Therefore, the present invention has advantages of simple structure and easy operation.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a partial diagram of an automated injection system according to a first embodiment of the present invention.

FIG. 2 is a partial diagram of an inserting assembly according to the first embodiment of the present invention.

FIG. 3 is a partial sectional diagram of the inserting assembly according to the first embodiment of the present invention.

FIG. 4 is a partial exploded diagram of the inserting assembly according to the first embodiment of the present invention.

FIG. 5 is a diagram illustrating a driving source is coupled to the inserting assembly according to the first embodiment of the present invention.

FIG. 6 is a diagram of a first ratchet component and a second ratchet component according to the first embodiment of the present invention.

FIG. 7 and FIG. 8 are diagrams of the inserting assembly in a first used state at different views according to the first embodiment of the present invention.

FIG. 9 and FIG. 10 are diagrams of the inserting assembly in a second used state at different views according to the first embodiment of the present invention.

FIG. 11 and FIG. 12 are diagrams of the inserting assembly in a third used state at different views according to the first embodiment of the present invention.

FIG. 13 is a diagram of a driving component and a driven component according to the first embodiment of the present invention.

FIG. 14 is a diagram illustrating a driving source is connected to an inserting assembly according to a second embodiment of the present invention.

FIG. 15 is a diagram of an inserting assembly according to a third embodiment of the present invention.

FIG. 16 is a diagram of an inserting assembly according to a fourth embodiment of the present invention.

### DETAILED DESCRIPTION

In the following detailed description of the preferred embodiments, reference is made to the accompanying drawings which form a part hereof, and in which is shown by way of illustration specific embodiments in which the invention may be practiced. In this regard, directional terminology, such as “top”, “bottom”, “left”, “right”, “front”, “back”, etc., is used with reference to the orientation of the Figure(s) being described. The components of the present invention can be positioned in a number of different orientations. As such, the directional terminology is used for purposes of illustration and is in no way limiting. Accordingly, the drawings and descriptions will be regarded as illustrative in nature and not as restrictive. Also, if not specified, the term “connect” or “coupled” is intended to mean either an indirect or direct electrical/mechanical connection. Thus, if a first device is connected or coupled to a second device, that connection may be through a direct electrical/mechanical connection, or through an indirect electrical/mechanical connection via other devices and connections.

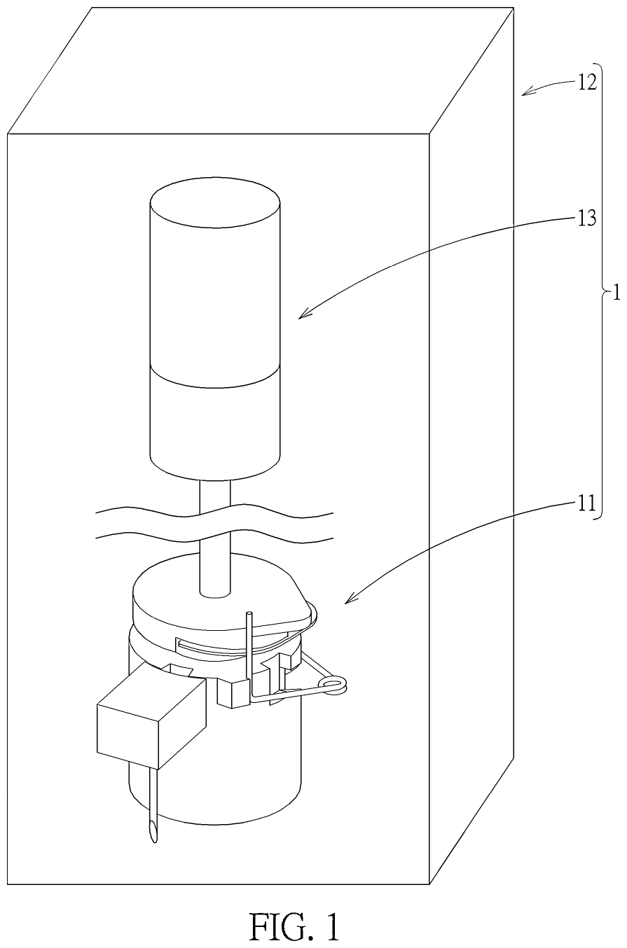

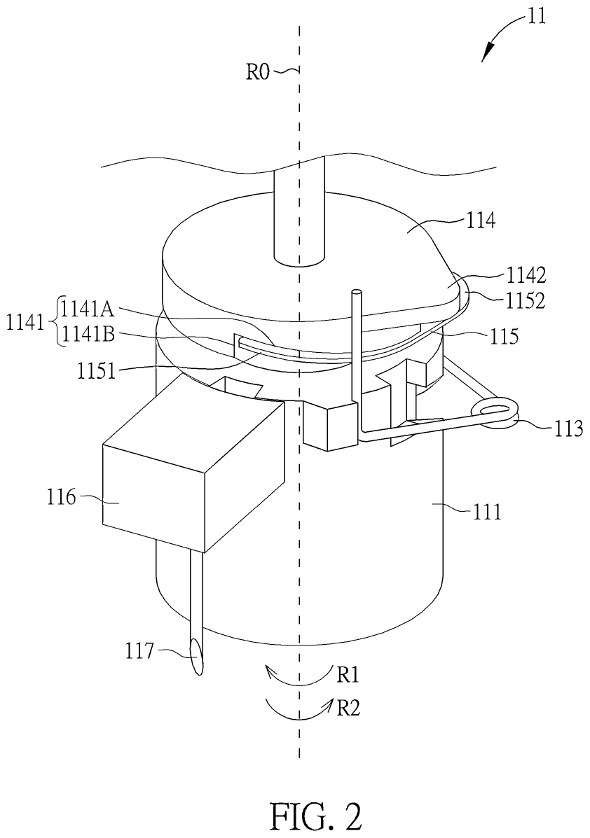

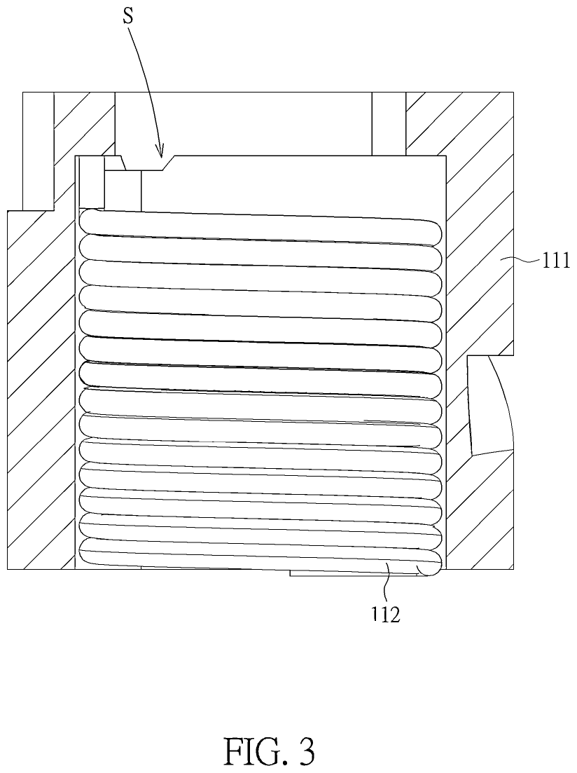

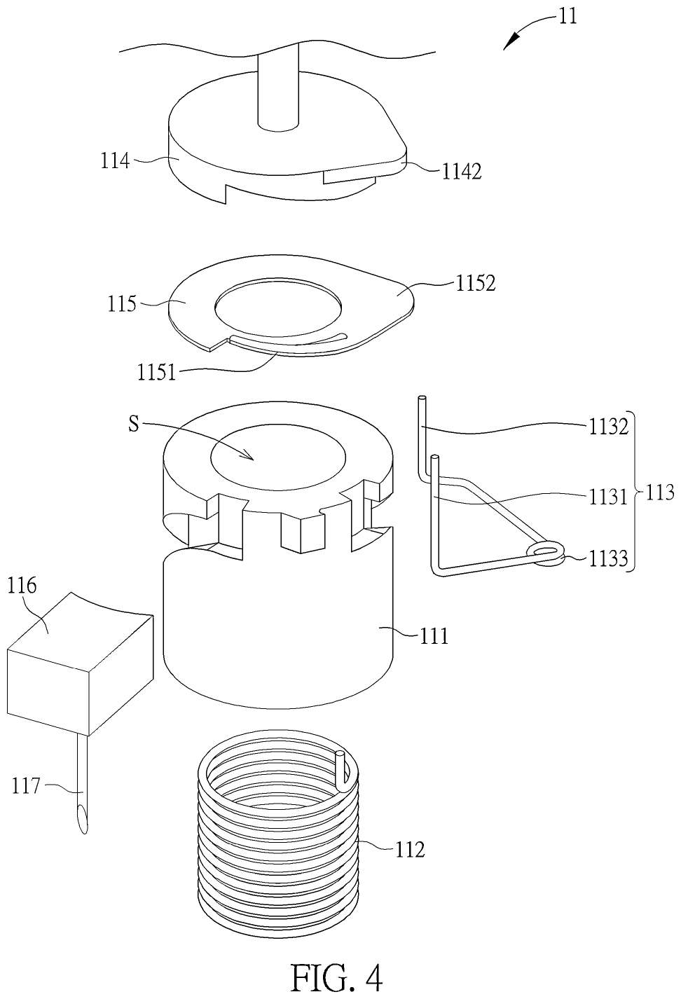

Please refer to FIG. 1 to FIG. 4. FIG. 1 is a partial diagram of an automated injection system 1 according to a first embodiment of the present invention. FIG. 2 is a partial diagram of an inserting assembly 11 according to the first embodiment of the present invention. FIG. 3 is a partial sectional diagram of the inserting assembly 11 according to the first embodiment of the present invention. FIG. 4 is a partial exploded diagram of the inserting assembly 11 according to the first embodiment of the present invention. As shown in FIG. 1 to FIG. 4, the automated injection system 1 includes the inserting assembly 11, a case 12 and a driving source 13. The inserting assembly 11 is mounted on the case 12. The driving source 13 is mounted on the case 12 and for driving the inserting assembly 11. The inserting assembly 11 includes a driving component 111, an actuating component 112, an abutting kit 113, a first ratchet component 114, a second ratchet component 115, a driven component 116 and an inserting component 117. The actuating component 112 is received in a space S of the driving component 111 and abuts against the driving component 111, so as to bias the driving component 111 to rotate around a rotating axis R0. The abutting kit 113 is configured to engage with the driving component 111 for stopping rotation of the driving component 111. The first ratchet component 114 is coaxially rotatable relative to the driving component 111. The driving source 13 is coupled to the first ratchet component 114, so as to drive the first ratchet component 114 to rotate. The second ratchet component 115 is coaxially rotatable relative to the driving component 111 and configured to be driven by the first ratchet component 114 to rotate. The abutting kit 113 is biased to engage with the driving component 111 for stopping rotation of the driving component 111 and is driven to disengage from the driving component 111 by the first ratchet component 114 or the second ratchet component 115 for allowing the actuating component 112 to drive the driving component 111 to rotate. The driven component 116 is movably engaged with the driving component 111, and the inserting component 117 is disposed on the driven component 116. The driven component 116 is configured to be driven by the driving component 111 to move when the driving component 111 is driven to rotate by the actuating component 112, and the inserting component 117 is movable together with the driven component 116 when the driven component 116 is driven to move by the driving component 111.

As shown in FIG. 1 to FIG. 3, in this embodiment, the driving source 13 and the driving component 111 can be an electric motor and a barrel, respectively, and the actuating component 112 and the inserting component 117 can be a torsional spring and a hollow needle, respectively. However, the present invention is not limited to this embodiment.

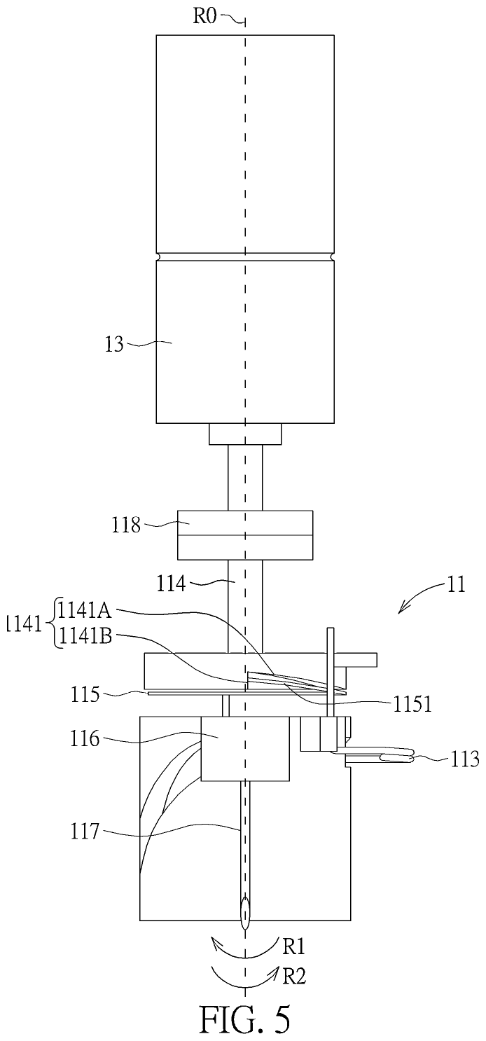

Please refer to FIG. 5. FIG. 5 is a diagram illustrating the driving source 13 is coupled to the inserting assembly 11 according to the first embodiment of the present invention. As shown in FIG. 5, the driving source 13 is coaxially connected to the first ratchet component 114 by a shaft coupling 118, e.g., a rigid coupling or a flexible coupling. However, the present invention is not limited to this embodiment. For example, in another embodiment, the driving source can be connected to the first ratchet component by a belt pulley drive mechanism or a gear drive mechanism.

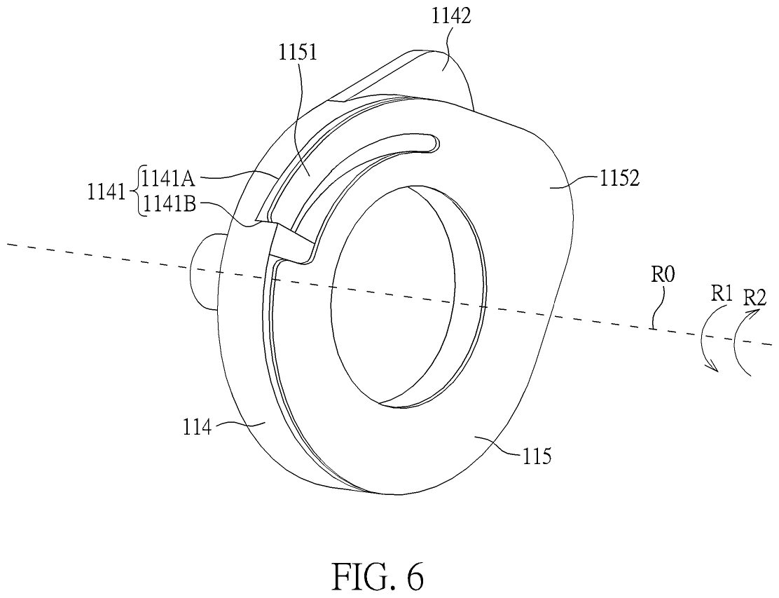

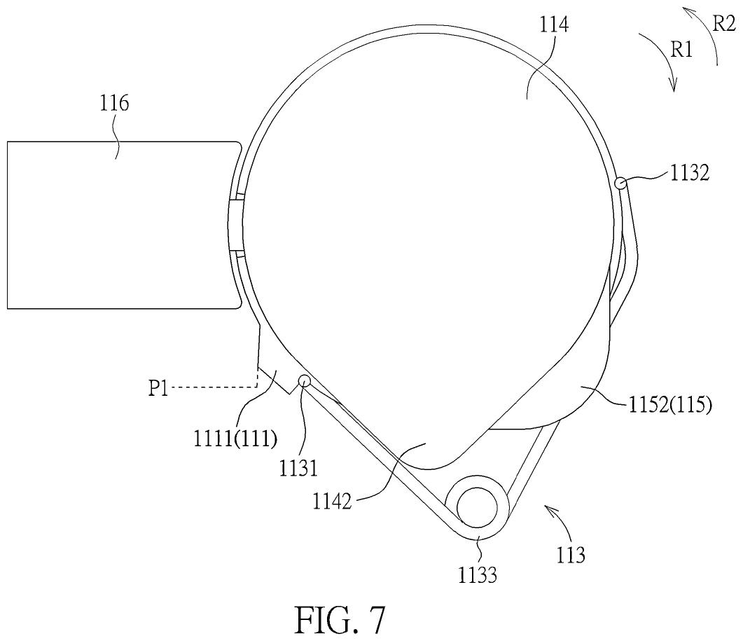

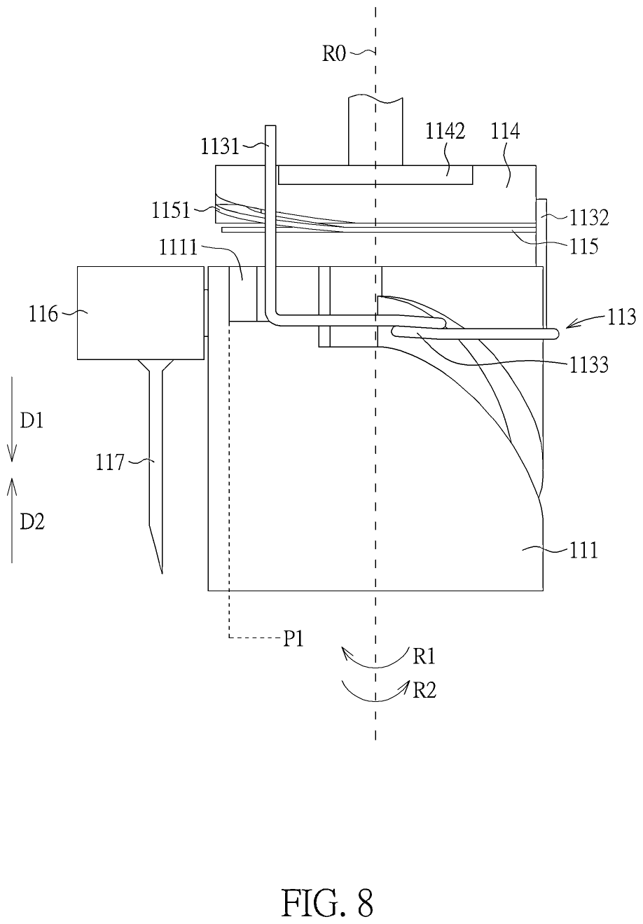

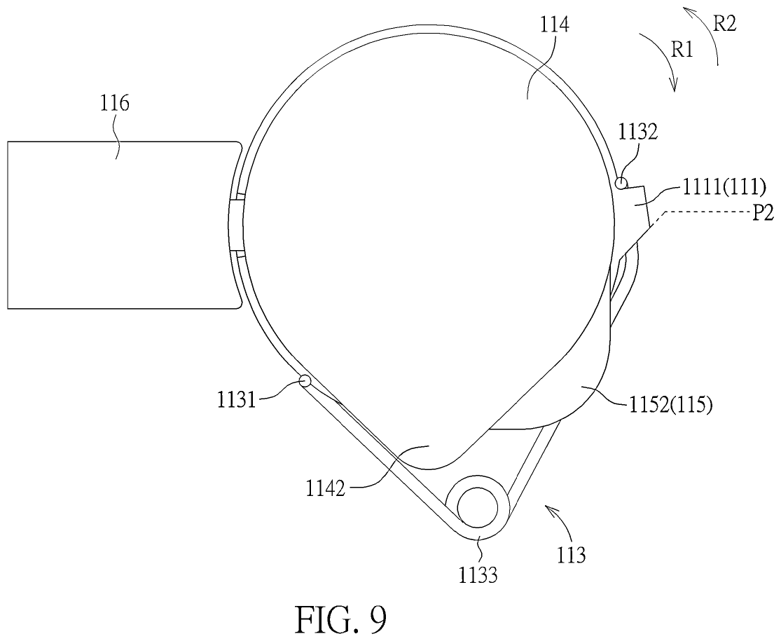

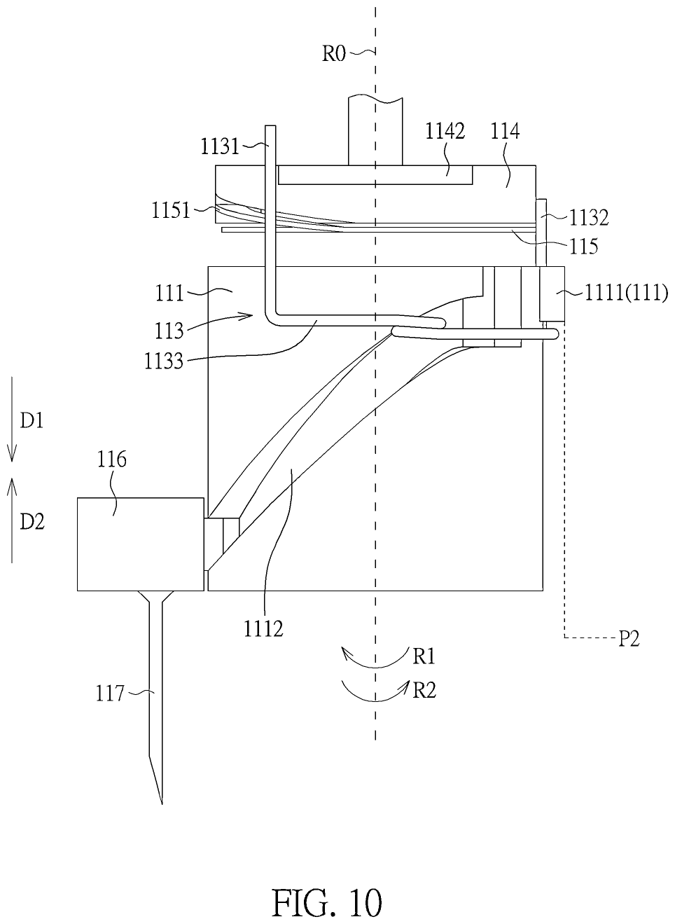

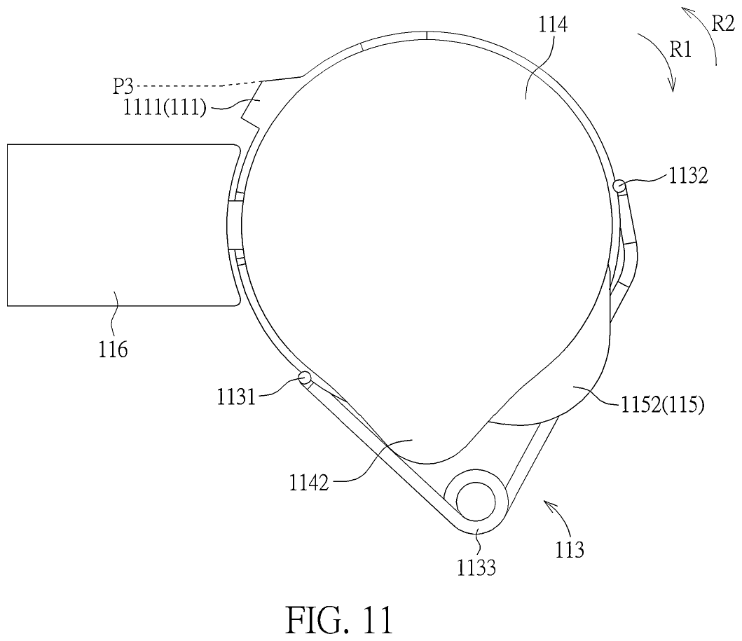

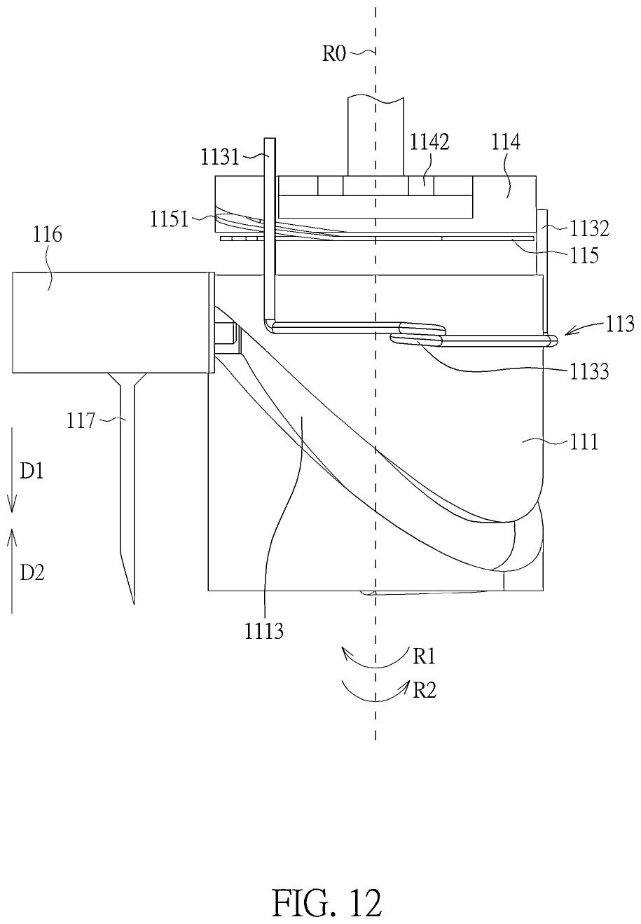

Please refer to FIG. 2 and FIG. 4 to FIG. 12. FIG. 6 is a diagram of the first ratchet component 114 and the second ratchet component 115 according to the first embodiment of the present invention. FIG. 7 and FIG. 8 are diagrams of the inserting assembly 11 in a first used state at different views according to the first embodiment of the present invention. FIG. 9 and FIG. 10 are diagrams of the inserting assembly 11 in a second used state at different views according to the first embodiment of the present invention. FIG. 11 and FIG. 12 are diagrams of the inserting assembly 11 in a third used state at different views according to the first embodiment of the present invention. As shown in FIG. 2 and FIG. 4 to FIG. 6, the second ratchet component 115 is resiliently deformed and not driven by the first ratchet component 114 when the first ratchet component 114 rotates along a first rotating direction R1, and the second ratchet component 115 is driven by the first ratchet component 114 to rotate together with the first ratchet component 114 along a second rotating direction R2 opposite to the first rotating direction R1 when the first ratchet component 114 rotates along the second rotating direction R2.

As shown in FIG. 2, FIG. 5 and FIG. 6, in this embodiment, a cooperating portion 1141 can be formed on the first ratchet component 114, and the second ratchet component 115 can include a resilient arm 1151 configured to cooperate with the cooperating portion 1141. When the first ratchet component 114 rotates along the first rotating direction R1, the resilient arm 1151 can be resiliently deformed by an inclined structure 1141A of the cooperating portion 1141, such that the second ratchet component 115 is not driven by the first ratchet component 114. When the first ratchet component 114 rotates along the second rotating direction R2, the resilient arm 1151 can be abutted by an erecting structure 1141B of the cooperating portion 1141, such that the second ratchet component 115 is driven by the first ratchet component 114 to rotate together with the first ratchet component 114 along the second rotating direction R2.

Furthermore, when the inserting assembly 11 is in the first used state as shown in FIG. 7 and FIG. 8, the actuating component 112 biases the driving component 111 to rotate along the second rotating direction R2 around the rotating axis R0, and the abutting kit 113 engages with the driving component 111 at a first position P1 for stopping the driving component 111 from rotating away from the first position P1 along the second rotating direction R2. When the first ratchet component 114 rotates along the first rotating direction R1, the first ratchet component 114 drives the abutting kit 113 to disengage from the driving component 111 at the first position P1 as shown in FIG. 7 and FIG. 8, such that the actuating component 112 drives the driving component 111 to rotate from the first position P1 as shown in FIG. 7 and FIG. 8 to a second position P2 as shown in FIG. 9 and FIG. 10 around the rotating axis R0 along the second rotating direction R2. As shown in FIG. 9 and FIG. 10, when the actuating component 112 drives the driving component 111 to rotate from the first position P1 to the second position P2 along the second rotating direction R2, the abutting kit 113 engages with the driving component 111 at the second position P2 for stopping the driving component 111 from rotating away from the second position P2 along the second rotating direction R2.

Afterwards, when the first ratchet component 114 rotates along the second rotating direction R2, the second ratchet component 115 is driven by the first ratchet component 114 to rotate together with the first ratchet component 114 along the second rotating direction R2, such that the second ratchet component 115 drives the abutting kit 113 to disengage from the driving component 111 at the second position P2. When the second ratchet component 115 drives the abutting kit 113 to disengage from the driving component 111 at the second position P2, the actuating component 112 drives the driving component 111 to rotate from the second position P2 as shown in FIG. 9 and FIG. 10 to a third position P3 as shown in FIG. 11 and FIG. 12 around the rotating axis R0 along the second rotating direction R2.

As shown in FIG. 2, FIG. 4 and FIG. 5, in this embodiment, the first ratchet component 114, the second ratchet component 115 and the driving component 111 are arranged along the rotating axis R0, and the second ratchet component 115 is located between the first ratchet component 114 and the driving component 111. The abutting kit 113 is located at a position corresponding to an outer periphery of the first ratchet component 114, an outer periphery of the second ratchet component 115, and an outer periphery of the driving component 111.

Furthermore, as shown in FIG. 4 to FIG. 12, the abutting kit 113 includes a first abutting portion 1131, a second abutting portion 1132 and a connecting portion 1133 integrally connected between the first abutting portion 1131 and the second abutting portion 1132 and sleeved on a mounting structure of the case 12, which is not shown in the figures. The first abutting portion 1131, the second abutting portion 1132 and the connecting portion 1133 can be made of resilient material. The first abutting portion 1131 is biased to cooperate with a stopping protrusion 1111 of the driving component 111 and a first pushing protrusion 1142 of the first ratchet component 114, and the second abutting portion 1132 is biased to cooperate with the stopping protrusion 1111 of the driving component 111 and a second pushing protrusion 1152 of the second ratchet component 115. Besides, in order to allow the first pushing protrusion 1142 to push the first abutting portion 1131 only, an end of the first abutting portion 1131 protrudes beyond an end of the second abutting portion 1132 along a direction from the second ratchet component 115 to the first ratchet component 114, i.e., the end of the second abutting portion 1132 is located at a position lower than the first pushing protrusion 1142 along a height direction parallel to the rotating axis R0.

As shown in FIG. 7 to FIG. 12, the driving component 111 drives the driven component 116 to move along an inserting direction D1 for driving the inserting component 117 to move together with the driven component 116 along the inserting direction D1 when the actuating component 112 drives the driving component 111 from the first position P1 to the second position P2. The driving component 111 drives the driven component 116 to move along a retracting direction D2 opposite to the inserting direction D1 for driving the inserting component 117 to move together with the driven component 116 along the retracting direction D2 when the actuating component 112 drives the driving component 111 to rotate from the second position P2 to the third position P3.

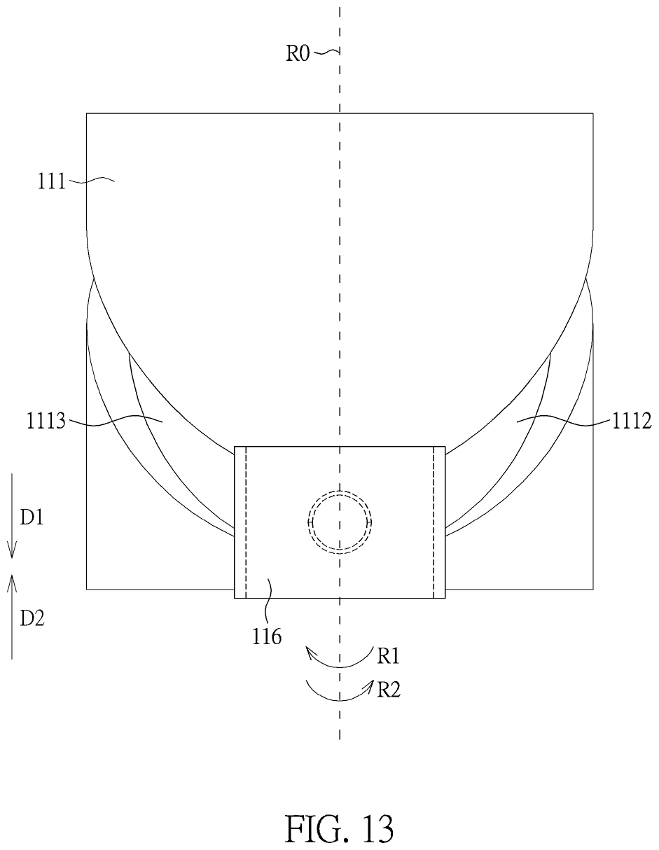

Please refer to FIG. 8, FIG. 10, FIG. 12 and FIG. 13. FIG. 13 is a diagram of the driving component 111 and the driven component 116 according to the first embodiment of the present invention. As shown in FIG. 8, FIG. 10, FIG. 12 and FIG. 13, in this embodiment, the driving component 111 includes an inserting inclined surface 1112 and a retracting inclined surface 1113. The inserting inclined surface 1112 and the retracting inclined surface 1113 are connected to each other. The inserting inclined surface 1112 is configured to abut against the driven component 116 for driving the driven component 116 to move along the inserting direction D1 when the actuating component 112 drives the driving component 111 to rotate away from the first position P1 and toward the second position P2 around the rotating axis R0, and the retracting inclined surface 1113 is configured to abut against the driven component 116 for driving the driven component 116 to move along the retracting direction D2 when the actuating component 112 drives the driving component 111 to rotate away from the second position P2 and toward the third position P3 around the rotating axis R0.

Detailed description for operational principle of the automated injection system 1 is provided as follows.

When it is desired to use the automated injection system 1, the driving source 13 can be activated to drive the first ratchet component 114 to rotate along the first rotating direction R1 to drive the first pushing protrusion 1142 to push the first abutting portion 1131 of the abutting kit 113 to move outwardly to disengage from the stopping protrusion 1111 of the driving component 111, for allowing the actuating component 112 to drive the driving component 111 to rotate from the first position P1 as shown in FIG. 7 and FIG. 8 to the second position P2 as shown in FIG. 9 and FIG. 10 around the rotating axis R0 along the second rotating direction R2. When the actuating component 112 drives the driving component 111 from the first position P1 to the second position P2, the driving component 111 drives the driven component 116 to move along the inserting direction D1 by a cooperation of the inserting inclined surface 1112 of the driving component 111 and the driven component 116 for driving the inserting component 117 to move together with the driven component 116 along the inserting direction D1, so as to achieve insertion of the inserting component 117 into a patient's skin. In addition, when the actuating component 112 reaches the second position P2 as shown in FIG. 9 and FIG. 10, the abutting kit 113 engages with the stopping protrusion 1111 of the driving component 111 for stopping the driving component 111 from rotating away from the second position P2 along the second rotating direction R2. It should be noticed that during the aforementioned process, the first ratchet component 114 can continuously rotate along the first rotating direction R1 or stop rotating after disengaging the first abutting portion 1131 of the abutting kit 113 from the stopping protrusion 1111 of the driving component 111.

Afterwards, the driving source 13 can be further operated to drive the first ratchet component 114 to rotate along the second rotating direction R2. When the first ratchet component 114 rotates along the second rotating direction R2, the second ratchet component 115 is driven by the first ratchet component 114 to rotate together with the first ratchet component 114 along the second rotating direction R2 to drive the second pushing protrusion 1152 of the second ratchet component 115 to push the second abutting portion 1132 of the abutting kit 113 to move outwardly to disengage from the stopping protrusion 1111 of the driving component 111, for allowing the actuating component 112 to drive the driving component 111 to rotate from the second position P2 as shown in FIG. 9 and FIG. 10 to the third position P3 as shown in FIG. 11 and FIG. 12 around the rotating axis R0 along the second rotating direction R2. When the actuating component 112 drives the driving component 111 from the second position P2 to the third position P3, the driving component 111 drives the driven component 116 to move along the retracting direction D2 by a cooperation of the retracting inclined surface 1113 of the driving component 111 and the driven component 116 for driving the inserting component 117 to move together with the driven component 116 along the retracting direction D2, so as to achieve retraction of the inserting component 117 out of the patient's skin. Besides, when the actuating component 112 reaches the third position P3 as shown in FIG. 11 and FIG. 12, the driven component 116 is abutted by an end portion of the retracting inclined surface 1113, such that the driving component 111 is stopped from rotating away from the third position P3 along the second rotating direction R2. It should be noticed that during the aforementioned process, the first ratchet component 114 can continuously rotate along the second rotating direction R2 or stop rotating after disengaging the second abutting portion 1132 of the abutting kit 113 from the stopping protrusion 1111 of the driving component 111.

Understandably, in another embodiment, the second ratchet component can be omitted, and in order to achieve retraction of the inserting component driven by the driving component, the abutting kit can be driven to disengage from the driving component at the second position by another mechanism rather than rotation of the first ratchet component along the second rotating component.

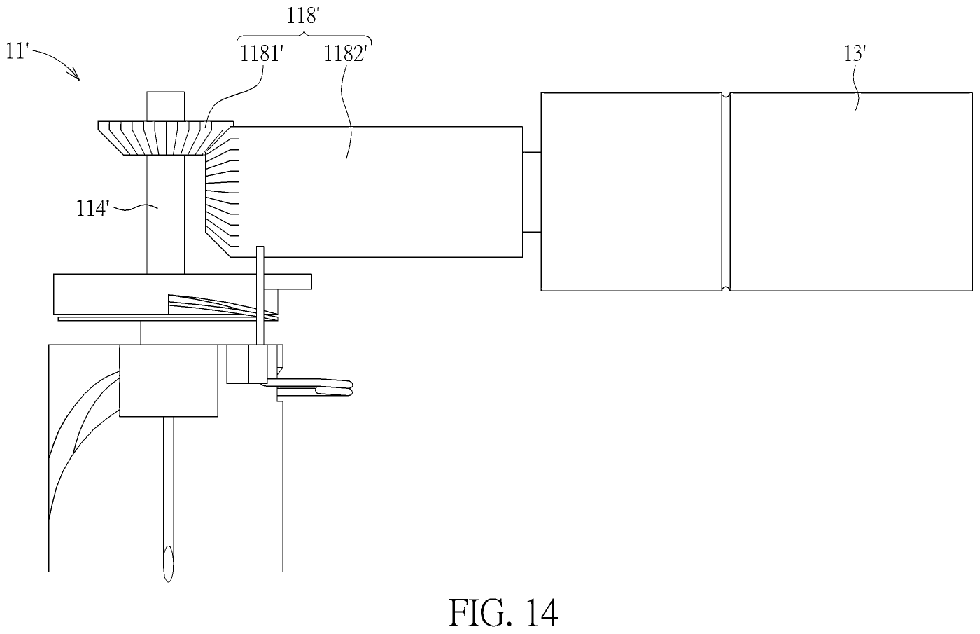

Please refer to FIG. 14. FIG. 14 is a diagram illustrating a driving source 13′ is connected to an inserting assembly 11′ according to a second embodiment of the present invention. The inserting assembly 11′ of the second embodiment is similar to the inserting assembly 11 of the first embodiment. As shown in FIG. 14, different from the first embodiment, in the second embodiment, a driving source 13′ is arranged perpendicularly to a rotating axis of a first ratchet component 114′ and connected to the first ratchet component 114′ by a gear drive mechanism 118′. The gear drive mechanism 118′ includes a first bevel gear 1181′ connected to the first ratchet component 114′ and a second bevel gear 1182′ connected to the driving source 13′ and rotatably engaged with the first bevel gear 1181′. Other details of the second embodiment are the same as the ones of the first embodiment. Detailed description is omitted herein for simplicity.

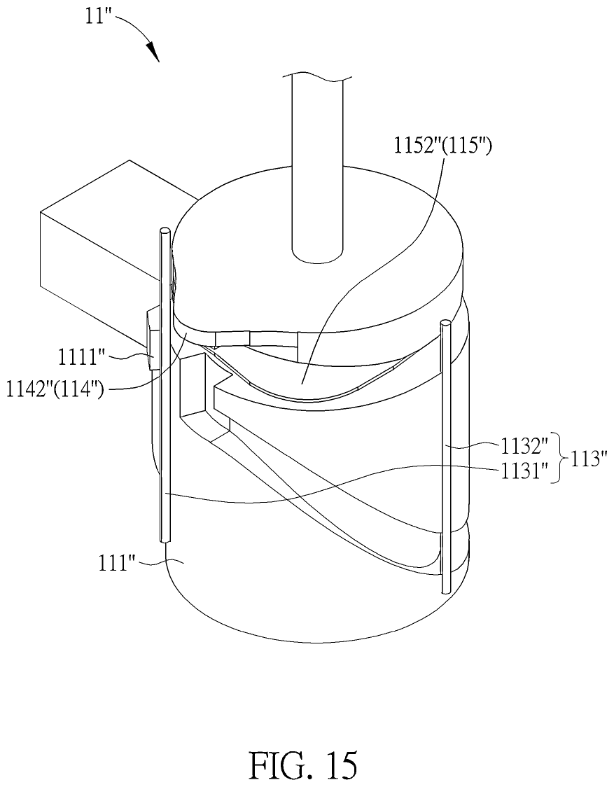

Please refer to FIG. 15. FIG. 15 is a diagram of an inserting assembly 11″ according to a third embodiment of the present invention. The inserting assembly 11″ of the third embodiment is similar to the inserting assembly 11 of the first embodiment. As shown in FIG. 15, different from the first embodiment, in the third embodiment, an abutting kit 113″ includes a first abutting portion 1131″ and a second abutting portion 1132″. The first abutting portion 1131″ and the second abutting portion 1132″ can be partially inserted into a mounting structure of the case, which is not shown in the figures, and separated from each other. The first abutting portion 1131″ and the second abutting portion 1132″ can be made of resilient material, so as to bias the first abutting portion 1131″ to cooperate with a stopping protrusion 1111″ of a driving component 111″ or a first pushing protrusion 1142″ of a first ratchet component 114″, and to bias the second abutting portion 1132″ to cooperate with the stopping protrusion 1111″ of the driving component 111″ or a second pushing protrusion 1152″ of a second ratchet component 115″. Other details of the third embodiment are the same as the ones of the first embodiment. Detailed description is omitted herein for simplicity.

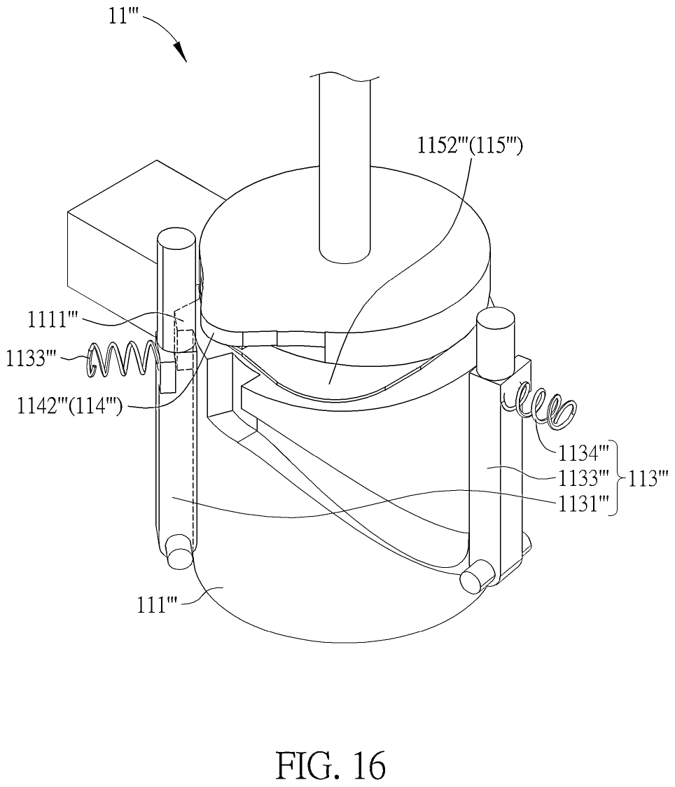

Please refer to FIG. 16. FIG. 16 is a diagram of an inserting assembly 11′″ according to a fourth embodiment of the present invention. The inserting assembly 11′″ of the fourth embodiment is similar to the inserting assembly 11 of the first embodiment. As shown in FIG. 16, different from the first embodiment, in the fourth embodiment, an abutting kit 113′″ includes a first abutting portion 1131′″, a second abutting portion 1132′″, a first recovering portion 1133′″ and a second recovering portion 1134′″. The first abutting portion 1131′″ and the second abutting portion 1132′″ can be pivotally disposed on a mounting structure of the case, which is not shown in the figures, and separated from each other. The first recovering portion 1133′″ is abutted between the first abutting portion 1131′″ and the case for biasing the first abutting portion 1131′″ to cooperate with a stopping protrusion 1111′″ of a driving component 111′″ or a first pushing protrusion 1142′″ of a first ratchet component 114′″. The second recovering portion 1134′″ is abutted between the second abutting portion 1132′″ and the case for biasing the second abutting portion 1132′″ to cooperate with the stopping protrusion 1111′″ of the driving component 111′″ or a second pushing protrusion 1152′″ of a second ratchet component 115′″. Other details of the fourth embodiment are the same as the ones of the first embodiment. Detailed description is omitted herein for simplicity.

In contrast to the prior art, the present invention can achieve the inserting component to move along the inserting direction and then along the retracting direction by rotating the first ratchet component along the first rotating direction and then along the second rotating direction only. Therefore, the present invention has advantages of simple structure and easy operation.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An inserting assembly comprising:
a driving component;
an actuating component abutting against the driving component and configured to bias the driving component to rotate around a rotating axis;
an abutting kit configured to engage with the driving component at a first position for stopping the driving component from rotating from the first position to a second position around the rotating axis;
a first ratchet component coaxially rotatable relative to the driving component, the first ratchet component driving the abutting kit to disengage from the driving component at the first position when the first ratchet component rotates along a first rotating direction, such that the actuating component drives the driving component to rotate from the first position to the second position;
a driven component movably engaged with the driving component, the driving component driving the driven component to move along an inserting direction when the actuating component drives the driving component from the first position to the second position; and
an inserting component disposed on the driven component and movable along the inserting direction together with the driven component.

2. The inserting assembly of claim 1, wherein the abutting kit is further configured to engage with the driving component at the second position for stopping the driving component from rotating away from the second position when the actuating component drives the driving component to rotate from the first position to the second position around the rotating axis.

3. The inserting assembly of claim 2, further comprising a second ratchet component coaxially rotatable relative to the driving component, the second ratchet component being resiliently deformed by the first ratchet component and not driven to rotate along the first rotating direction by the first ratchet component when the first ratchet component rotates along the first rotating direction, and the second ratchet component being driven by the first ratchet component to rotate along a second rotating direction opposite to the first rotating direction when the first ratchet component rotates along the second rotating direction.

4. The inserting assembly of claim 3, wherein the second ratchet component drives the abutting kit to disengage from the driving component at the second position when the second ratchet component is driven by the first ratchet component to rotate along the second rotating direction, and the actuating component drives the driving component to rotate away from the second position around the rotating axis when the second ratchet component drives the abutting kit to disengage from the driving component at the second position.

5. The inserting assembly of claim 4, wherein the driving component drives the driven component to move along a retracting direction when the actuating component drives the driving component to rotate away from the second position around the rotating axis, and the driven component drives the inserting component to move together with the driven component along the retracting direction when the driving component drives the driven component to move along the retracting direction.

6. The inserting assembly of claim 5, wherein the driving component comprises an inserting inclined surface and a retracting inclined surface, the inserting inclined surface and the retracting inclined surface are connected to each other, the inserting inclined surface is configured to abut against the driven component for driving the driven component to move along the inserting direction when the actuating component drives the driving component to rotate away from the first position and toward the second position around the rotating axis, and the retracting inclined surface is configured to abut against the driven component for driving the driven component to move along the retracting direction when the actuating component drives the driving component to rotate away from the second position and toward a third position around the rotating axis.

7. The inserting assembly of claim 4, wherein the first ratchet component, the second ratchet component and the driving component are arranged along the rotating axis, the second ratchet component is located between the first ratchet component and the driving component, and the abutting kit is located at a position corresponding to an outer periphery of the first ratchet component, an outer periphery of the second ratchet component, and an outer periphery of the driving component.

8. The inserting assembly of claim 7, wherein the abutting kit comprises a first abutting portion and a second abutting portion, an end of the first abutting portion protrudes beyond an end of the second abutting portion along a direction from the second ratchet component to the first ratchet component, the first abutting portion is configured to cooperate with the driving component and the first ratchet component, and the second abutting portion is configured to cooperate with the driving component and the second ratchet component.

9. The inserting assembly of claim 8, wherein the abutting kit further comprises a connecting portion integrally connected between the first abutting portion and the second abutting portion, and the first abutting portion, the second abutting portion and the connecting portion are made of resilient material.

10. The inserting assembly of claim 8, wherein the first abutting portion and the second abutting portion are separated from each other and made of resilient material.

11. The inserting assembly of claim 8, wherein the abutting kit further comprises a first recovering portion and a second recovering portion, the first recovering portion and the second recovering portion are made of resilient material and abut against the first abutting portion and the second abutting portion, respectively.

12. The inserting assembly of claim 1, wherein the actuating component biases the driving component to rotate along a second rotating direction opposite to the first rotating direction around the rotating axis.

13. An inserting assembly comprising:
a driving component;
an actuating component abutting against the driving component and configured to bias the driving component to rotate around a rotating axis;
an abutting kit configured to engage with the driving component;
a first ratchet component coaxially rotatable relative to the driving component, the first ratchet component driving the abutting kit to disengage from the driving component at a first position when the first ratchet component rotates along a first rotating direction, such that the actuating component drives the driving component to rotate from the first position to a second position around the rotating axis along a second rotating direction opposite to the first rotating direction, and the abutting kit engaging with the driving component at the second position for stopping the driving component from rotating away from the second position along the second rotating direction when the actuating component drives the driving component to rotate from the first position to the second position along the second rotating direction;
a second ratchet component coaxially rotatable relative to the driving component, the second ratchet component being driven by the first ratchet component to rotate along the second rotating direction when the first ratchet component rotates along the second rotating direction, the second ratchet component driving the abutting kit to disengage from the driving component at the second position when the second ratchet component is driven by the first ratchet component to rotate along the second rotating direction, and the actuating component drives the driving component to rotate from the second position to a third position around the rotating axis along the second rotating direction when the second ratchet component drives the abutting kit to disengage from the driving component at the second position;
a driven component movably engaged with the driving component, the driving component driving the driven component to move along an inserting direction when the actuating component drives the driving component from the first position to the second position, the driving component driving the driven component to move along a retracting direction when the actuating component drives the driving component to rotate from the second position to the third position; and
an inserting component disposed on the driven component and movable along the inserting direction or the retracting direction together with the driven component.

14. An automated injection system comprising:
a case; and
an inserting assembly mounted on the case, and the inserting assembly comprising:
a driving component;
an actuating component abutting against the driving component and configured to bias the driving component to rotate around a rotating axis;
an abutting kit configured to engage with the driving component at a first position for stopping the driving component from rotating from the first position to a second position around the rotating axis;
a first ratchet component coaxially rotatable relative to the driving component, the first ratchet component driving the abutting kit to disengage from the driving component at the first position when the first ratchet component rotates along a first rotating direction, such that the actuating component drives the driving component to rotate from the first position to the second position;
a driven component movably engaged with the driving component, the driving component driving the driven component to move along an inserting direction when the actuating component drives the driving component to rotate from the first position to the second position; and
an inserting component disposed on the driven component and movable along the inserting direction together with the driven component.

15. The automated injection system of claim 14, wherein the abutting kit is further configured to engage with the driving component at the second position for stopping the driving component from rotating away from the second position when the actuating component drives the driving component to rotate from the first position to the second position around the rotating axis, the inserting assembly further comprises a second ratchet component coaxially rotatable relative to the driving component, the second ratchet component is resiliently deformed by the first ratchet component and not driven to rotate along the first rotating direction by the first ratchet component when the first ratchet component rotates along the first rotating direction, and the second ratchet component is driven by the first ratchet component to rotate along a second rotating direction opposite to the first rotating direction when the first ratchet component rotates along the second rotating direction.

16. The automated injection system of claim 15, wherein the second ratchet component drives the abutting kit to disengage from the driving component at the second position when the second ratchet component is driven by the first ratchet component to rotate along the second rotating direction, the actuating component drives the driving component to rotate away from the second position around the rotating axis when the second ratchet component drives the abutting kit to disengage from the driving component at the second position, the driving component drives the driven component to move along a retracting direction when the actuating component drives the driving component to rotate away from the second position around the rotating axis, and the driven component drives the inserting component to move together with the driven component along the retracting direction when the driving component drives the driven component to move along the retracting direction.

17. The automated injection system of claim 16, wherein the driving component comprises an inserting inclined surface and a retracting inclined surface, the inserting inclined surface and the retracting inclined surface are connected to each other, the inserting inclined surface is configured to abut against the driven component for driving the driven component to move along the inserting direction when the actuating component drives the driving component to rotate away from the first position and toward the second position around the rotating axis, and the retracting inclined surface is configured to abut against the driven component for driving the driven component to move along the retracting direction when the actuating component drives the driving component to rotate away from the second position and toward a third position around the rotating axis.

18. The automated injection system of claim 16, wherein the first ratchet component, the second ratchet component and the driving component are arranged along the rotating axis, the second ratchet component is located between the first ratchet component and the driving component, and the abutting kit is located at a position corresponding to an outer periphery of the first ratchet component, an outer periphery of the second ratchet component, and an outer periphery of the driving component.

19. The automated injection system of claim 18, wherein the abutting kit comprises a first abutting portion and a second abutting portion, an end of the first abutting portion protrudes beyond an end of the second abutting portion along a direction from the second ratchet component to the first ratchet component, the first abutting portion is configured to cooperate with the driving component and the first ratchet component, and the second abutting portion is configured to cooperate with the driving component and the second ratchet component.

20. The automated injection system of claim 14, wherein the actuating component biases the driving component to rotate along the second rotating direction around the rotating axis.
